SEQUENCE LISTING (9 sequences)

SEQ ID NO: 1 - protein, 449 aa, Mouse
MGSDVRDLNALLPAVSSLGGGGGGCGLPVSGARQWAPVLDFAPPGASAYGSLGGPAPPPAPPPPPPPPHSFIKQEPSWGGAEPHEEQCLSAFTLHFSGQFTGTAGACRYGPFGPPPPSQASSGQARMFPNAPYLPSCLESQPTIRNQGYSTVTFDGAPSYGHTPSHHAAQFPNHSFKHEDPMGQQGSLGEQQYSVPPPVYGCHTPTDSCTGSQALLLRTPYSSDNLYQMTSQLECMTWNQMNLGATLKGMAAGSSSSVKWTEGQSNHGIGYESENHTAPILCGAQYRIHTHGVFRGIQDVRRVSGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWHSCQKKFARSDELVRHHNMHQRNMTKLHVAL

SEQ ID NO: 2 - protein, 449 aa, Human
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 3 - protein, 8 aa, Artificial  Sequence
  Synthetic  Peptide
GASAYGSL

SEQ ID NO: 4 - protein, 8 aa, Artificial  Sequence
  Synthetic  Peptide
CNKRYFKL

SEQ ID NO: 5 - protein, 9 aa, Artificial  Sequence
  Synthetic  Peptide
RMFPNAPYL

SEQ ID NO: 6 - protein, 9 aa, Artificial  Sequence
  Synthetic  Peptide
YSSDNLYQM

SEQ ID NO: 7 - protein, 9 aa, Artificial  Sequence
  Synthetic  Peptide
CMTWNQMNL

SEQ ID NO: 8 - protein, 9 aa, Artificial  Sequence
  Synthetic  Peptide
SLGEQQYSV

SEQ ID NO: 9 - protein, 9 aa, Artificial  Sequence
  Synthetic  Peptide
CYTWNQMNL

(19) United States
(12) Patent Application Publication
(10) Pub. No.: US 2006/0165708 A1
(43) Pub. Date: Jul. 27, 2006

(54) Cancer vaccine containing cancer antigen based on tumor suppressor gene wt1 product and cationic liposomes

(75) Inventors: Tadanori Mayumi, Kobe-shi, Hyogo (JP); Haruo Sugiyama, Osaka (JP); Yoshiyuki Ohsugi, Tokyo (JP)

Correspondence Address: MORRISON & FOERSTER LLP, 1650 TYSONS BOULEVARD, SUITE 300, MCLEAN, VA, 22102, US

(73) Assignee: Chugai Seiyaku Kabushiki Kaisha, Tokyo (JP)

(21) Appl. No.: 10/482,327

(22) PCT Filed: Jun. 28, 2002
(86) PCT No.: PCT/JP02/06597

§ 371 Date: Dec. 29, 2003

(30) Foreign Application Priority Data
Jun. 29, 2001 (JP) 2001-199449

(51) Int. Cl. A61K 39/00

(52) U.S. Cl. 424/185.1

(57) ABSTRACT

A cancer vaccine comprising a cancer antigen which comprises, as an active ingredient, the product of a tumor suppressor gene WT1, a partial peptide or a modified version thereof, and a cationic liposome.

### FIELD OF THE INVENTION

The present invention relates to a cancer vaccine comprising a cancer antigen based on the product of a tumor suppressor gene WT1 of Wilms tumor and lipofectin. This cancer vaccine is useful as an anti-cancer vaccine for blood cancers such as leukemia, myelodysplastic syndrome, multiple myeloma and malignant lymphoma, or solid cancers such as gastric cancer, colon cancer, lung cancer, breast cancer, germ cell cancer, liver cancer, skin cancer, bladder cancer, prostatic cancer, uterine cancer, cervical cancer and ovarian cancer, as well as all other cancers that express WT1.

### BACKGROUND ART

Immunological mechanisms for eliminating foreign substances generally comprise the humoral immunity which involves macrophages that recognize antigen so as to function as antigen presenting cells, helper T cells that recognize the antigen presentation by said macrophages and produce various lymphokines so as to activate other T cells etc., and B lymphocytes, etc., that differentiate into antibody-producing cells by the action of said lymphokines; and the cellular immunity in which killer T cells differentiated by antigen presentation attack and destroy target cells.

At present, cancer immunity is mainly derived from cellular immunity which involves killer T cells. In killer T cell-mediated cancer immunity, precursor T cells that recognized cancer antigen presented in the form of a complex with the major histocompatibility complex (MHC) class I differentiate and grow to produce killer T cells, which attack and destroy cancer cells. At this time, cancer cells have presented the complex of the MHC class I antigen and cancer antigen on the cell surface, which becomes the target for killer T cells (Curr. Opin. Immuno. 5:709, 1993; Curr. Opin. Immunol. 5:719, 1993; Cell 82:13, 1995; Immunol. Rev. 146:167, 1995).

The above cancer antigen presented on the target cancer cells by MHC class I antigen is believed to be a peptide composed of about 8-12 amino acids produced as a result of processing by intracellular protease of antigen proteins synthesized in the cancer cells (Curr. Opin. Immunol. 5:709, 1993; Curr. Opin. Immunol. 5:719, 1993; Cell 82:13, 1995; Immunol. Rev. 146:167, 1995).

The tumor suppressor gene WT1 (WT1 gene) of Wilms tumor was isolated from chromosome 11p13 as one of the causative genes for Wilms tumor based on the analysis of the WAGR syndrome that is accompanied by Wilms tumor, aniridia, urogenital abnormality, mental retardation etc. (Gessler, M. et al., Nature, Vol. 343, pp. 774-778, 1990), and its genomic DNA is about 50 kb comprising ten exons and its cDNA is about 3 kb. The amino acid sequence deduced from the cDNA is as set forth in SEQ ID NO: 1 (Mol. Cell. Biol. 11:1707, 1991).

The WT1 gene is highly expressed in human leukemia, and the treatment of leukemic cells with a WT1 antisense oligomer leads to the suppression of the cell growth (Japanese Unexamined Patent Publication (Kokai) No. 9-104627), which suggests that the WT1 gene is acting on the growth of leukemic cells in a facilitative manner. Furthermore, the WT1 gene has also been highly expressed in solid cancers such as gastric cancer, colon cancer, lung cancer, breast cancer, germ cell cancer, liver cancer, skin cancer, bladder cancer, prostatic cancer, uterine cancer, cervical cancer and ovarian cancer (Japanese Patent Application No. 9-191635), and the WT1 gene was found to be a new tumor marker for leukemia and solid cancers.

Thus, it is expected that the administration of a peptide having about 8-12 amino acids comprising a portion of expression products of the WT1 gene could serve as a cancer vaccine against the above range of cancers. However, the administration of such a peptide as it is cannot serve as a cancer vaccine. This is because it is expected that the peptide administered cannot be effectively delivered to the major histocompatibility complex class I on the antigen-presenting cells.

Lipofectin, a cationic liposome, is a 1:1 mixture of an artificial lipid N-[1-(2,3-dioleyloxy)propyl]-N,N,N-trimethylammonium chloride (DOTMA) and a phospholipid dioleoylphosphatidylethanolamine (DOPE), and attracted attention as a nonviral carrier for introducing genes. Subsequently, attention was given to the fact that it can serve to deliver peptide antigens to the major histocompatibility complex class I on the antigen-presenting cells (Rinsho Menneki 34(6):842-847, 2000). However, the degree of versatility of cationic liposomes as carriers for peptide antigens is unknown, and it is not known either whether they can serve as carriers for cancer antigen peptides comprising fragments of expression products of the tumor suppressor gene WT1 gene.

### DISCLOSURE OF THE INVENTION

Thus, the present invention provides a novel cancer vaccine comprising a cancer antigen peptide derived from a WT1 gene expression product and a substance useful as a carrier therefor.

After intensive and extensive research in order to solve the above problems, the present inventors have confirmed that, in the amino acid sequence of expression product of the WT1 gene, a polypeptide comprising 7-30 contiguous amino acids containing at least one amino acid that is estimated to serve as an anchor amino acid serves as a cancer antigen in the binding with the mouse and human MHC class I and MHC class II, and that cationic liposomes such as lipofectin are useful as carriers for this peptide antigen and, thereby, have completed the present invention.

Thus, the present invention provides a cancer vaccine comprising a cancer antigen containing a mouse WT1 gene expression product or a portion thereof, and a cationic liposome. In a preferred embodiment, the present invention provides a cancer vaccine comprising a cancer antigen that comprises as an active ingredient a peptide comprising 6-30 amino acids containing at least one amino acid selected from the group consisting of Phe, Tyr, Leu, Met, Asn and Ile, that is estimated to function as an anchor amino acid for binding to the MHC antigen, in the amino acid sequence as set forth in SEQ ID NO: 1 corresponding to the cDNA of the MHC antigen, and a cationic liposome.

Furthermore, the present invention provides a cancer vaccine comprising a cancer antigen that comprises as an active ingredient a peptide comprising 7-30 amino acids containing at least one amino acid selected from the group consisting of Met, Leu, and Val, that is estimated to function as an anchor amino acid for binding to the MHC antigen, in an amino acid sequence as set forth in SEQ ID NO: 2 corresponding to the cDNA of human WT1, and a cationic liposome.

### BRIEF EXPLANATION OF THE DRAWINGS

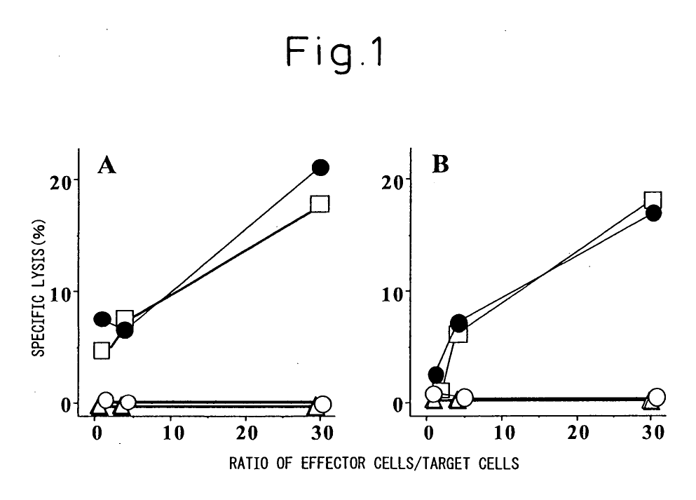

In FIG. 1, A is a graph that compares the ability of inducing cytotoxic T cells of a mixture (closed circle) of a cancer antigen peptide D^{b} 126 and lipofectin (LPF), a lipopolysaccharide-blast (open square) pulsed with D^{b} 126, lipofectin alone (open triangle) and the cancer antigen peptide D^{b} 126 alone (open circle) using (3) RNAS cells and (4) RNAS cells stimulated with the cancer antigen peptide D^{b} 126, and B is a graph of the result in which tests similar to the above A were carried out using (1) C1498 cells and (2) WT1 gene-introduced C1498 cells. A indicates that the combination of the cancer antigen peptide D^{b} 126 and lipofectin has an activity of inducing cytotoxic T cells, and B indicates that the activity thereof is WT1-specific.

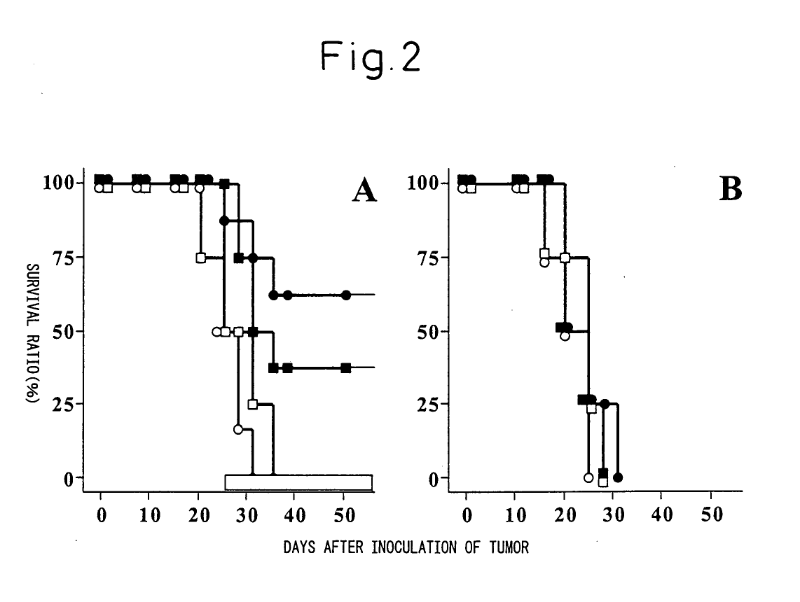

In FIG. 2, A is a graph that shows the effect of lipofectin as an adjuvant (carrier) for the anti-cancer effect of the peptide D^{b} 126 using WT1 gene-introduced C1498 cells, and B is a graph that shows the result of similar tests using C1498 cells. Signs that indicate the test substances are the same as in FIG. 1. A indicates that lipofectin is effective as an adjuvant (carrier) for the cancer antigen peptide D^{b} 126, and the comparison of A and B shows that the anti-cancer effect is WT1-specific.

### EMBODIMENT FOR CARRYING OUT THE INVENTION

In accordance with the present invention, as a basis for designing cancer antigen peptides, K^{b} and D^{b} of mouse MHC class I as well as A0201 of human HLA were selected, and peptides estimated to have a high affinity with them were selected.

Based on the description in Immunogenetics 41:178-228 (1995), Phe and Try at position 5 as well as Leu and Met etc. at position 8 are expected to be the anchor amino acids for binding to K^{b}, and Asn at position 5 as well as Met and Ile etc. at position 9 are expected to be the anchor amino acids for binding to D^{b}.

It is also known that the size of cancer antigen peptides presented on the surface of cancer cells by MHC class I is about 8-12 amino acids. Thus, the cancer antigen peptide of the present invention is a peptide comprising 7-30 contiguous amino acids containing at least one amino acid of Phe, Tyr, Leu, Met, Asn and Ile in the amino acid sequence of the WT1 gene product as set forth in SEQ ID NO: 1. The number of amino acids is preferably 8-12, for example 8 or 9.

In accordance with the present invention, specific embodiments include, as a peptide that binds to K^{b} of MHC class I, the following peptides comprising 8 amino acids:

| | |
|---|---|
| K^{b} 45 | |
| Gly Ala Ser Ala Tyr Gly Ser Leu | (SEQ ID NO: 3) |
| K^{b} 330 | |
| Cys Asn Lys Arg Tyr Phe Lys Leu, | (SEQ ID NO: 4) |
| and, | |

as a peptide that binds to D^{b} of MHC class I, the following peptides comprising 9 amino acids:

| | |
|---|---|
| D^{b} 126 | |
| Arg Met Phe Pro Asn Ala Pro Tyr Leu | (SEQ ID NO: 5) |
| D^{b} 221 | |
| Tyr Ser Ser Asp Asn Leu Tyr Gln Met | (SEQ ID NO: 6) |
| D^{b} 235 | |
| Cys Met Thr Trp Asn Gln Met Asn Leu. | (SEQ ID NO: 7) |

In the above sequences, the underlined amino acids are those that are thought to serve as anchors.

All of them have strong to moderate binding affinities (Kd values) for K^{b} or D^{b}, and the D^{b} 126 peptide having the highest binding affinity was used in the following experiments.

For humans, based on the description in Immunogenetics 41:178-228 (1995), Leu and Met at position 2 from the N-terminal and Val and Leu at position 9 from the N-terminal are expected to be anchor amino acids for binding to human HLA-A0201. Thus, from among the amino acid sequence of human WT1 protein (Mol. Cell. Biol. 11:1707-1712, 1991) (SEQ ID NO: 2), the following two peptides:

D^{b} 126

(SEQ ID NO: 5)

Arg Met Phe Pro Asn Ala Pro Tyr Leu
(the same as D^{b} 126 in mice)
WH 187

(SEQ ID NO: 8)

Ser Leu Gly Glu Gln Gln Tyr Ser Val
(the underlined are anchor amino acids)

comprising nine amino acids are mentioned as complying with the above condition.

The cancer antigen peptide of the present invention may also be a peptide in which a modification such as amino acid substitution has been introduced into a peptide which is a portion of the expression product of the WT1 gene. As an example of such a modified peptide, there can be mentioned a cancer antigen peptide comprising as an active ingredient a peptide that comprises 9-30 amino acids containing the following amino acid sequence: Cys Tyr Thr Trp Asn Gln Met Asn Leu (SEQ ID NO: 9). As a specific embodiment, there can be mentioned a peptide having an amino acid sequence: Cys Tyr Thr Trp Asn Gln Met Asn Leu (SEQ ID NO: 9) in which Met at position 2 of the above peptide D^{b} 235 (SEQ ID NO: 7) has been changed to Tyr.

As a cationic liposome, there can be mentioned a liposome comprising N-[1-(2,3-dioleyloxy)propyl]-N,N,N-trimethylammonium chloride (DOTMA), N-[1-(2,3-dioleyloxy)propyl]-N,N,N-trimethylammonium methyl sulfate (DOTAP) or dioctadecylamide-glycylspermine (DOGS), or mixtures thereof with a neutral lipid.

As a neutral lipid, there can be mentioned, for example, licithin, lysolecithin, sphingomyelin, phosphatidic acid, phosphatidylethanolamine, and dioleoylphosphatidylethanolamine (DOPE). As an example of mixtures, there can be mentioned lipofectin which is a 1:1 mixture of an artificial lipid N-[1-(2,3-dioleyloxy)propyl]-N,N,N-trimethylammonium chloride (DOTMA) and a phospholipid dioleoylphosphatidylethanolamine (DOPE).

The cancer vaccine of the present invention can be used for the prevention or treatment of cancers that are accompanied by increased levels of the WT1 gene expression, for example blood cancers such as leukemia, myelodysplastic syndrome, multiple myeloma and malignant lymphoma, and solid cancers such as gastric cancer, colon cancer, lung cancer, breast cancer, germ cell cancer, liver cancer, skin cancer, bladder cancer, prostatic cancer, uterine cancer, cervical cancer and ovarian cancer. This vaccine can be administered by oral administration, or parenteral administration such as intraperitoneal, cutaneous, dermal, intramuscular, intravenous, and nasal administration.

The dosage of the cancer vaccine of the present invention is generally 0.1 μg to 1 mg/kg per day.

### EXAMPLES

The usefulness of the cancer vaccine of the present invention will now be explained with reference to Examples.

### Example 1

Preparation of Lipopolysaccharide-Blast (LPS-blast)

From C57BL/6 mice, spleen cells were recovered, and the cells were incubated for 3 days in a complete RPMI medium containing lipopolysaccharide (LPS) (10 μg/ml). After washing, the cells were incubated in a complete RPMI medium containing the cancer antigen peptide D^{b} 126 (1 μM) and ovalbumin (OVA) (100 μg/ml). After washing, the cells were suspended in 2 ml Hanks, balanced salt solution (HBSS) which was set as the lipopolysaccharide-blast (LPS-blast).

Evaluation of the Ability of Inducing Cytotoxic T Cells (CTL)

C57BL/6 mice were immunized three times weekly by subcutaneous administration, to the back thereof, of a mixture of the cancer antigen peptide D^{b} 126 and lipofectin (LPF) (mixed at a 1:2 weight ratio of D^{b} 126 and LPF), and as a positive control by the intraperitoneal administration of lipopolysaccharide-blast (LPS-blast) (1 ml/mouse). Ten days after the final immunization, spleen cells were recovered and set as effector cells. Spleen cells stimulated with the cancer antigen peptide D^{b} 126 (1 μM, 2 hours, 37° C., 5% CO2) were washed with HBSS to obtain stimulator cells.

The above effector cells (5×106 cells/well) and the above stimulator cells (2.5×106 cells/well) were mixed, and then subjected to lymphocyte-lymphocyte mixed culture for the in vitro second challenge of the cytotoxic T cells. Five days later, cytotoxic T cells were recovered. (1) C1498 cells, (2) C1498 cells that have introduced therein and express the WT1 gene (C1498muWT1), (3) RMA-S cells, and (4) RMA-S cells stimulated with the cancer antigen D^{b} 126 (1 μM, treated for 1 hour at 5% CO2) (D^{b} 126-pulsed RMA-S), each labelled with Na₂⁵¹CrO₄ (0.56 MBq/10⁶ cells, treated for 1 hour at 37° C. and 5% CO2), were plated as the target cells onto 96-well microtiter plates (10⁴ cells/well), to which cytotoxic T cells prepared as above were plated. Effector cells were added thereto, cultured for 4 hours, and the radioactivity of ⁵¹Cr liberated into the supernatant was counted. Cytotoxic activity was calculated according to the following equation:

$$Cell lysis ( \% ) = \frac{( experimental release - spontaneous release )}{( maximum release - spontaneous release )} \times 100$$

Results

First, in order to examine whether or not the induced cytotoxic T cells are specific for the cancer antigen peptide D^{b} 126, the above (3) RMA-S cells and (4) D^{b} 126-pulsed RMA-S cells were used as the target cells, and as a result the induction of cytotoxic T cells specific for the cancer antigen peptide D^{b} 126 was confirmed (FIG. 1, A). Furthermore, in order to examine whether or not the induced cytotoxic T cells specifically damage WT1-expressing cells, the above (1) C1498 cells and (2) the WT1 gene-introduced cells, C1498muWT1, were used, and as a result the induction of WT1-specific CTL was confirmed (FIG. 1, B).

### Example 2

Cancer Antigen-Specific Anti-Tumor Effect when Lipofectin (LPF) was Used as the Cancer Vaccine Carrier

Since Example 1 has shown that cytotoxic T cells are effectively induced by using lipofectin (LPF) as an adjuvant for the cancer antigen peptide D^{b} 126, cancer antigen-specific anti-tumor effect when immunized using lipofectin as an adjuvant (carrier) was examined for the purpose of further confirming the usefulness of lipofectin (LPF) as an adjuvant for cancer vaccines.

As the tumor model, WT1 gene-introduced C1498 cells (C1498muWT1 cells) were used; as the immunization animal, C57BL/6 mice were used; and as the model cancer antigen, the peptide D^{b} 126 was used. Thus, C57BL/6 mice were immunized three times weekly by subcutaneous administration, to the back thereof, of the same mixture as in Example 1 of the cancer antigen peptide D^{b} 126 and lipofectin (LPF) (10 nmol/mouse), or by the intraperitoneal administration of lipopolysaccharide-blast (LPS-blast) (1 ml), and one week after the final immunization C1498muWT1 cells or C1498 cells were intraperitoneally transplanted at an amount of 2×10⁶ cells/100 ml. The effect of tumor vaccine was determined daily, and was evaluated by calculating tumor size using the following equation:
[Tumor size]=[(long diameter)×(short diameter)²]^{1/3}

In each group, the experiment was terminated when tumor size reached 20 mm.

Results

The evaluation of lipofectin (LPF) as an adjuvant (carrier) for cancer vaccine was carried out using WT1 as the model tumor antigen and WT1 gene-introduced cells (C1498muWT1 cells) as the model tumor, and using, as an index, resistance against C1498muWT1 cells when the peptide D^{b} 126/lipofectin (LPF) mixture was used for immunization. As a result, when the peptide D^{b} 126/lipofectin (LPF) mixture was used for immunization, complete rejection was observed in three out of eight cases (FIG. 2, A).

Furthermore, in order to confirm that this anti-tumor effect is WT1-specific, a similar study was carried out using C1498 cells that are not expressing WT1. As a result, there were no differences seen from the non-immunized group in any of the cases in which (a) peptide D^{b} 126/lipofectin (LPF) mixture, (b) free peptide D^{b} 126, and (c) lipopolysaccharide-blast (LPS-blast) were immunized (FIG. 2, B). Therefore, it was confirmed that the above anti-tumor effect is WT1-specific.

1. A cancer vaccine comprising a cancer antigen which comprises as an active ingredient the product of a tumor suppressor gene WT1, a partial peptide or a modified version thereof, and a cationic liposome.

2. The cancer vaccine according to claim 1 wherein said cancer antigen is a peptide comprising 7-30 contiguous amino acids containing at least one anchor amino acid selected from Phe, Tyr, Leu, Met, Asn and Ile in the amino acid sequence as set forth in SEQ ID NO: 1, or a peptide comprising 7-30 contiguous amino acids containing at least one anchor amino acid selected from Met, Leu and Val in the amino acid sequence as set forth in SEQ ID NO: 2.

3. The cancer vaccine according to claim 1 or 2 wherein said antigen is a cancer antigen that permits a high expression of the tumor suppressor gene WT1.

4. The cancer vaccine according to claim 1 or 2 wherein said cancer is leukemia, myelodysplastic syndrome, malignant lymphoma, multiple myeloma, gastric cancer, colon cancer, lung cancer, breast cancer, germ cell cancer, liver cancer, skin cancer, bladder cancer, prostatic cancer, uterine cancer, cervical cancer or ovarian cancer.

5. The cancer vaccine according to any of claims 1 to 4 wherein said cancer antigen peptide is any of:

| | |
|---|---|
| Kb 45 | |
| Gly Ala Ser Ala Tyr Gly Ser Leu | (SEQ ID NO: 3) |
| Kb 330 | |
| Cys Asn Lys Arg Tyr Phe Lys Leu | (SEQ ID NO: 4) |
| D^{b} 126 | |
| Arg Met Phe Pro Asn Ala Pro Tyr Leu | (SEQ ID NO: 5) |
| Db 221 | |
| Tyr Ser Ser Asp Asn Leu Tyr Gln Met | (SEQ ID NO: 6) |
| Db 235 | |
| Cys Met Thr Trp Asn Gln Met Asn Leu, | (SEQ ID NO: 7) |
| and | |
| WH 187 | |
| Ser Leu Gly Glu Gln Gln Tyr Ser Val. | (SEQ ID NO: 8) |

6. The cancer vaccine according to claim 5 wherein said cancer antigen peptide is:
(SEQ ID NO: 5)

| | |
|---|---|
| D^{b} 126 | Arg Met Phe Pro Asn Ala Pro Tyr Leu, or |

(SEQ ID NO: 8)

| | |
|---|---|
| WH 187 | Ser Leu Gly Glu Gln Gln Tyr Ser Val. |

7. The cancer vaccine according to claim 1 wherein said cancer antigen peptide is a peptide comprising 9-30 amino acids containing the following amino acid sequence: Cys Tyr Thr Trp Asn Gln Met Asn Leu (SEQ ID NO: 9).

8. The cancer vaccine according to claim 7 wherein said cancer antigen peptide is a peptide comprising the following amino acid sequence: Cys Tyr Thr Trp Asn Gln Met Asn Leu (SEQ ID NO: 9).

9. The cancer vaccine according to any one of claims 1 to 8 wherein said cationic liposome is a liposome comprising N-[1-(2,3-dioleyloxy)propyl]-N,N,N-trimethylammonium chloride, N-[1-(2,3-dioleyloxy)propyl]-N,N,N-trimethylammonium methyl sulfate or dioctadecylamide-glycylspermine; or a mixture thereof with a neutral lipid.

10. The cancer vaccine according to claim 9 wherein said cationic liposome is lipofectin.

11. The use of a cancer antigen which comprises as an active ingredient the product of the tumor suppressor gene WT1, a partial peptide or a modified version thereof, and a cationic liposome for the production of cancer vaccine.

12. The use according to claim 11 wherein said cancer antigen is a peptide comprising 7-30 contiguous amino acids containing at least one anchor amino acid selected from Phe, Tyr, Leu, Met, Asn and Ile in the amino acid sequence as set forth in SEQ ID NO: 1, or a peptide comprising 7-30 contiguous amino acids containing at least one anchor amino acid selected from Met, Leu and Val in the amino acid sequence as set forth in SEQ ID NO: 2.

13. The use according to claim 11 or 12 wherein said antigen is a cancer antigen that permits high expression of the tumor suppressor gene WT1.

14. The use according to claim 11 or 12 wherein said cancer is leukemia, myelodysplastic syndrome, malignant lymphoma, multiple myeloma, gastric cancer, colon cancer, lung cancer, breast cancer, germ cell cancer, liver cancer, skin cancer, bladder cancer, prostatic cancer, uterine cancer, cervical cancer or ovarian cancer.

15. The use according to any of claims 11 to 14 wherein said cancer antigen peptide is any of:

| | |
|---|---|
| Kb 45 | |
| Gly Ala Ser Ala Tyr Gly Ser Leu | (SEQ ID NO: 3) |
| Kb 330 | |
| Cys Asn Lys Arg Tyr Phe Lys Leu | (SEQ ID NO: 4) |
| D^{b} 126 | |
| Arg Met Phe Pro Asn Ala Pro Tyr Leu | (SEQ ID NO: 5) |
| Db 221 | |
| Tyr Ser Ser Asp Asn Leu Tyr Gln Met | (SEQ ID NO: 6) |
| Db 235 | |
| Cys Met Thr Trp Asn Gln Met Asn Leu, | (SEQ ID NO: 7) |
| and | |
| WH 187 | |
| Ser Leu Gly Glu Gln Gln Tyr Ser Val. | (SEQ ID NO: 8) |

16. The use according to claim 15 wherein said cancer antigen peptide is:
(SEQ ID NO: 5)

| | |
|---|---|
| D^{b} 126 | Arg Met Phe Pro Asn Ala Pro Tyr Leu, or |

(SEQ ID NO: 8)

| | |
|---|---|
| WH 187 | Ser Leu Gly Glu Gln Gln Tyr Ser Val. |

17. The use according to claim 11 wherein said cancer antigen peptide is a peptide comprising 9-30 amino acids containing the following amino acid sequence: Cys Tyr Thr Trp Asn Gln Met Asn Leu (SEQ ID NO: 9).

18. The use according to claim 17 wherein said cancer antigen peptide is a peptide comprising the following amino acid sequence: Cys Tyr Thr Trp Asn Gln Met Asn Leu (SEQ ID NO: 9).

19. The use according to any one of claims 11 to 18 wherein said cationic liposome is a liposome comprising N-[1-(2,3-dioleyloxy)propyl]-N,N,N-trimethylammonium chloride, N-[1-(2,3-dioleyloxy)propyl]-N,N,N-trimethylammonium methyl sulfate or dioctadecylamide-glycylspermine; or a mixture thereof with a neutral lipid.

20. The use according to claim 19 wherein said cationic liposome is lipofectin.

21. A method of treating patients with cancer, said method comprising administering a cancer antigen which comprises as an active ingredient the product of the tumor suppressor gene WT1, a partial peptide or a modified version thereof, and a cationic liposome.

22. The method according to claim 21 wherein said cancer antigen is a peptide comprising 7-30 contiguous amino acids containing at least one anchor amino acid selected from Phe, Tyr, Leu, Met, Asn and Ile in the amino acid sequence as set forth in SEQ ID NO: 1, or a peptide comprising 7-30 contiguous amino acids containing at least one anchor amino acid selected from Met, Leu and Val in the amino acid sequence as set forth in SEQ ID NO: 2.

23. The method according to claim 21 or 22 wherein said antigen is a cancer antigen that permits high expression of the tumor suppressor gene WT1.

24. The method according to claim 21 or 22 wherein said cancer is leukemia, myelodysplastic syndrome, malignant lymphoma, multiple myeloma, gastric cancer, colon cancer, lung cancer, breast cancer, germ cell cancer, liver cancer, skin cancer, bladder cancer, prostatic cancer, uterine cancer, cervical cancer or ovarian cancer.

25. The method according to any of claims 11 to 24 wherein said cancer antigen peptide is any of:

| | |
|---|---|
| Kb 45 | |
| Gly Ala Ser Ala Tyr Gly Ser Leu | (SEQ ID NO: 3) |
| Kb 330 | |
| Cys Asn Lys Arg Tyr Phe Lys Leu | (SEQ ID NO: 4) |
| D^{b} 126 | |
| Arg Met Phe Pro Asn Ala Pro Tyr Leu | (SEQ ID NO: 5) |
| Db 221 | |
| Tyr Ser Ser Asp Asn Leu Tyr Gln Met | (SEQ ID NO: 6) |
| Db 235 | |
| Cys Met Thr Trp Asn Gln Met Asn Leu, | (SEQ ID NO: 7) |
| and | |
| WH 187 | |
| Ser Leu Gly Glu Gln Gln Tyr Ser Val. | (SEQ ID NO: 8) |

26. The method according to claim 25 wherein said cancer antigen peptide is:

| | |
|---|---|
| D^{b} 126 | |
| Arg Met Phe Pro Asn Ala Pro Tyr Leu, | (SEQ ID NO: 5) |
| or | |
| WH 187 | |
| Ser Leu Gly Glu Gln Gln Tyr Ser Val. | (SEQ ID NO: 8) |

27. The method according to claim 21 wherein said cancer antigen peptide is a peptide comprising 9-30 amino acids containing the following amino acid sequence: Cys Tyr Thr Trp Asn Gln Met Asn Leu (SEQ ID NO: 9).

28. The method according to claim 27 wherein said cancer antigen peptide is a peptide comprising the following amino acid sequence: Cys Tyr Thr Trp Asn Gln Met Asn Leu (SEQ ID NO: 9).

29. The method according to any one of claims 21 to 28 wherein said cationic liposome is a liposome comprising N-[1-(2,3-dioleyloxy)propyl]-N,N,N-trimethylammonium chloride, N-[1-(2,3-dioleyloxy)propyl]-N,N,N-trimethylammonium methyl sulfate or dioctadecylamide-glycylspermine; or a mixture thereof with a neutral lipid.

30. The method according to claim 29 wherein said cationic liposome is lipofectin.
